(19) United States
(12) Patent Application Publication
(10) Pub. No.: US 2010/0330093 A1
(43) Pub. Date: Dec. 30, 2010

(54) TREATMENT OF MELANOMA WITH ALPHA THYMOSIN PEPTIDES IN COMBINATION WITH ANTIBODIES AGAINST CYTOTOXIC T LYMPHOCYTE-ASSOCIATED ANTIGEN 4 (CTLA4)

(75) Inventors: Israel Rios, Menlo Park, CA (US); Cynthia W. Tuthill, Menlo Park, CA (US)

Correspondence Address: COOLEY LLP;ATTN: Patent Group, Suite 1100, 777 - 6th Street, NW, WASHINGTON, DC, 20001, US

(73) Assignee: SciClone Pharmaceuticals, Inc., Foster City, CA (US)

(21) Appl. No.: 12/747,817

(22) PCT Filed: Dec. 8, 2008
(86) PCT No.: PCT/US08/13480

§ 371 Date: Aug. 20, 2010

Related U.S. Application Data
(60) Provisional application No. 61/013,101, filed on Dec. 12, 2007.

(51) Int. Cl. A61K 39/395; A61P 35/00; A61P 35/04

(52) U.S. Cl. 424/142.1; 424/173.1

(57) ABSTRACT

Melanoma or a metastasis thereof is treated in a human patient in a combination therapy which includes administering a melanoma-treating combination to a human melanoma patient during a treatment regimen, the combination including an alpha thymosin peptide and antibodies against cytotoxic T lymphocyte-associated antigen 4 (CTLA4).

### CROSS-REFERENCE TO RELATED APPLICATIONS

This application claims the benefit of U.S. Provisional Application No. 61/013,101, filed Dec. 12, 2007, the disclosure of which is incorporated herein in its entirety by reference.

### FIELD OF THE INVENTION

The present invention relates to the field of melanoma treatment.

### BACKGROUND OF THE INVENTION

Skin cancer is the most common form of cancer in the United States. In 2007, The American Cancer Society estimates that approximately 8,110 deaths will occur from melanoma and another 59,940 cases of melanoma are expected to be diagnosed in this country.

Melanoma is a malignant tumor of melanocytes which are found predominantly in skin but also in bowel and the eye (uveal melanoma). It is one of the rarer types of skin cancer but causes the majority of skin cancer related deaths.

The treatment includes surgical removal of the tumor; adjuvant treatment; chemo- and immunotherapy, or radiation therapy. Of particular danger are metastases of the primary melanoma tumor.

There remains a need in the art for improved treatments of melanoma.

### SUMMARY OF THE INVENTION

In accordance with one embodiment of the present invention, a method of treating melanoma or a metastasis thereof in a human patient is a combination therapy which comprises administering a melanoma-treating combination to a human melanoma patient during a treatment regimen, the combination comprising an alpha thymosin peptide and antibodies against cytotoxic T lymphocyte-associated antigen 4 (CTLA4).

### DESCRIPTION OF THE PREFERRED EMBODIMENTS

The present invention is directed to a method of treating melanoma or metastases thereof in human patients. In one embodiment, the method involves administering a melanoma-treating effective combination to human melanoma patients, the combination comprising an alpha thymosin peptide and antibodies against cytotoxic T lymphocyte-associated antigen 4 (CTLA4).

In certain embodiments, the combination further includes one or more additional agents to combat or treat melanoma.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1. Suitable dosages of the alpha thymosin peptide can be within the range of about 0.001-10 mg/kg/day.

The terms “thymosin alpha 1” and “TA1” refer to peptides having the amino acid sequence disclosed in U.S. Pat. No. 4,079,137, the disclosure of which is incorporated herein by reference.

Thymosin alpha 1 (TA1), initially isolated from Thymosin Fraction 5 (TF5), has been sequenced and chemically synthesized. TA1 is a 28 amino acid peptide with a molecular weight of 3108.

Effective amounts of an alpha thymosin peptide are amounts which may be dosage units within a range corresponding to about 0.1-20 mg of TA1, or about 1-10 mg of TA1, or about 2-10 mg of TA1, or about 2-7 mg of TA1. The dosage unit may be within a range of 3-6.5 mg, and may comprise about 1.6, 3.2 or 6.4 mg of TA1, or about 3.2 or about 6.4 mg of TA1. A dosage unit may be administered once per day, or a plurality of times per day.

Melanoma has various stages, which may include Stage 0, I, II, III and IV, as well as their respective subdivisions. In certain embodiments, the melanoma being treated is malignant metastatic melanoma. In certain embodiments, the melanoma being treated is stage I, stage II, stage III or stage IV. In other embodiments, the melanoma being treated is stage M1a, M1b or M1c melanoma. Dacarbazine (DTIC) is the conventional chemotherapeutic drug for the patients with melanoma.

The alpha thymosin peptide may be administered in a treatment regimen which includes administration to the patient of antibodies against cytotoxic T lymphocyte-associated antigen 4 (CTLA4). These include, without limitation, 9H10 (EBIOSCIENCE), MDX010 (MEDAREX), 1F4 (GENETEX), BNI3 (GENETEX), Q01 (ABNOVA), A01 (ABNOVA), M08 (ABNOVA), 1B8 (ABCAM), WKH203 (ABCAM), ab9984 (ABCAM), ab13486 (ABCAM), ipilimumab, ticilimumab or a combination thereof.

9H10 is a functional-grade hamster anti-mouse antibody against CTLA-4 on T cells.

The method of the present invention may comprise administering the alpha thymosin peptide along with administering antibodies against cytotoxic T lymphocyte-associated antigen 4 (CTLA4), during a course of the treatment regimen. The CTLA4 antibodies may be administered concurrently with the alpha thymosin peptide or separately therefrom during the treatment regimen, e.g., on the same day(s) as the alpha thymosin peptide or on different days during the course of the treatment regimen. In certain embodiments, the CTLA4 antibodies are administered in a dosage range of, e.g., 0.001-50 mg/kg patient body weight per day of administration, or about 0.01-20 mg/k, or about 1-15 mg/kg.

As noted above, in certain embodiments, the combination further includes one or more additional agents to combat or treat melanoma. Such additional agents may be antineoplastic agents such as alkylating antineoplastic agents (AIkAA), which include, without limitation, dacarbazine (DTIC). Additional agent(s) of the combination, such as alkylating antineoplastic agents (AIkAA), may be administered to patient within a dosage range of, e.g., about 700-1300 mg/m²/day, more preferably in a dosage range of about 800-1200 mg/m²/day, and most preferably about 1000 mg/m²/day.

The various components of the combination may be administered concurrently with, or separately from, other components in a treatment regimen.

In certain embodiments, the treatment regimen comprises a plurality of days, with the alpha thymosin peptide comprising thymosin alpha 1 (TA1), and the TA1 being administered to the patient during at least a portion of the treatment regimen at a dosage within a range of about 0.5-10 mg/day. In certain embodiments, the dosage is within a range of about 1.5-7 mg/day, or within a range of about 1.6-6.4 mg/day. In certain embodiments, the dosage is within a range of 1.7-10 mg/day, or about 1.7-7 mg/day, or about 3-7 mg/day. Exemplary dosages include 1.6, 3.2 and 6.4 mg/day.

In certain embodiments, the treatment regimen comprises administering the alpha thymosin peptide for a period of about 1-10 days, followed by about 1-5 days of non-administration of the alpha thymosin peptide. The alpha thymosin peptide may be administered daily for about 3-5 days, followed by about 2-4 days of non-administration of the alpha thymosin peptide. The alpha thymosin peptide may be administered daily for about 4 days, followed by about 3 days of non-administration of the alpha thymosin peptide.

According to one embodiment, the invention comprises use of an alpha thymosin peptide and CTLA4 antibodies in manufacture of a melanoma-treating effective pharmaceutical combination or medicament for use in a treatment regimen for treating melanoma or a metastasis thereof in a human melanoma patient.

According to one embodiment, said medicament is for use in a treatment regimen which substantially excludes any immune-stimulating cytokine to said patient during said treatment regimen in an amount significant for treatment of melanoma or a metastasis thereof.

According to one embodiment, said LDH blood level is below 475 IU/L.

According to one embodiment, said LDH blood level is between 100-335 IU/L.

One embodiment is the manufacture of a pharmaceutical combination including said alpha thymosin peptide, said combination further comprising CTLA4 antibodies for use during a course of the treatment regimen, which alpha thymosin peptide and CTLA4 antibodies may be administered separately or together.

According to one embodiment, said CTLA4 antibodies comprise ipilimumab.

According to one embodiment, said CTLA4 antibodies comprise ticilimumab.

According to one embodiment, said CTLA4 antibodies comprise 9H10.

According to one embodiment, said CTLA4 antibodies comprise MDX010.

According to one embodiment, said CTLA4 antibodies comprise Q01.

According to one embodiment, said CTLA4 antibodies comprise A01.

According to one embodiment, said CTLA4 antibodies comprise M08.

According to one embodiment, said CTLA4 antibodies comprise 1B8.

According to one embodiment, said CTLA4 antibodies comprise WKH203.

According to one embodiment, said CTLA4 antibodies comprise ab9984.

According to one embodiment, said CTLA4 antibodies comprise ab13486.

According to one embodiment, said medicament is for use in a treatment regimen which comprises a plurality of days, said alpha thymosin peptide comprises thymosin alpha 1 (TA1), and said TA1 is for use in administration to said patient during at least a portion of said treatment regimen at a dosage within a range of 0.5-10 mg/day.

According to one embodiment, said dosage is within a range of 1.5-7 mg/day.

According to one embodiment, said dosage is 3.2 mg/day.

According to one embodiment, said dosage is 6.4 mg/day.

According to one embodiment, said alpha thymosin peptide is TA1 and said medicament is for use in a treatment regimen which comprises administration of TA1 daily for a period of about 1-10 days, followed by about 1-5 days of non-administration of said TA1.

According to one embodiment, said TA1 is for use in administration daily for about 3-5 days, followed by about 2-4 days of non-administration of said TA1.

According to one embodiment, said TA1 is for use in administration daily for about 4 days, followed by about 3 days non-administration of said TA1.

### Example 1

### 9H10

C57BL/6 mice were implanted subcutaneously with murine B16 melanoma cells (Cell Culture Center, Institute of Basic Medical Sciences, Peking Union Medical College and Chinese Academy of Medical Sciences PUMC&CAMS, Beijing, P.R.China), followed by treatment with TA-1 or DTIC alone or in combination for 14 consecutive days. The day of tumor implantation was defined as Day 0. Meanwhile 9H10 was given in two groups via i.p. on Day 3, 6 and 9. TA-1 and DTIC were administered daily by s.c. injection. In total, 5 groups of mice (n=10) were used: Group 1: vehicle; Group 2: DTIC 5 mg/kg; Group 3: TA-1 6 mg/kg; Group 4: DTIC 5 mg/kg+9H10 (Day 3 (100 μg), Day 6 (50 μg), Day 9 (50 μg)); Group 5: DTIC 5 mg/kg+TA-1 6 mg/kg+9H10 (Day 3 (100 μg), Day 6 (50 μg), Day 9 (50 μg)) (Table 1). The animals were six weeks old and weighed between 18 and 22 grams at the start of the study. Tumor volume and body weight were measured every three days, and tumor weights were measured on Day 16 at the end of the study.

TABLE 1
Treatment Regimen and Study Design

| Group | Group | | Number of | Dosing | Necropsy |
|---|---|---|---|---|---|
| Number | Name | Treatment | Animals | Period | Day |
| 1 | Vehicle | PBS | 10 | Days 3-15 | Day 16 |
| 2 | DTIC | DTIC, 5 mg/kg, s.c., daily | 10 | | |
| 3 | TA-1 | TA-1, 6 mg/kg, s.c., daily | 10 | | |
| 4 | 9H10 + | 9H10, i.p., Day 3 (100 μg), | 10 | | |
| | DTIC | Day 6 (50 μg), Day 9 (50 μg) + | | | |
| | | DTIC, 5 mg/kg, s.c., daily | | | |
| 5 | 9H10 + | 9H10, i.p., Day 3 (100 μg), | 10 | | |
| | DTIC + | Day 6 (50 μg), Day 9 (50 μg) + | | | |
| | TA-1 | DTIC, 5 mg/kg, s.c., daily + | | | |
| | | TA-1, 6 mg/kg, s.c., daily | | | |

Phosphate buffered saline (PBS) was used as the negative control article (vehicle). The test article TA-1 was dissolved in PBS to achieve the proper dose concentration as indicated in Table 2. TA-1 solution was stored at 2-8° C. and used up in one week. DTIC(SIGMA) was initially dissolved in 0.01 N HCl and then diluted with PBS to 1 mg/mL. DTIC dosing solution was kept on ice, protected from light, and used within one day. Final dosing solutions were prepared on the day of use by diluting the stock solution to 1× with water. The antibody reagent 9H10 (EBIOSCIENCE) was a ready-to-use agent at a concentration of 1 mg/mL.

TABLE 2
Dose Formulation

| | | Dose | |
|---|---|---|---|
| | Dose level | Volume | Concentration |
| Treatment | (mg/kg) | (mL/kg) | (mg/mL) |
| TA-1 | 6 | 5 | 1.2 |
| DTIC | 5 | 5 | 1.0 |

Mortality and moribundity were checked twice daily, the body weights were recorded once every 3 days, and tumors were measured using a caliper once every 3 days. At the end of the study (Day 16), the animals were euthanized by CO₂ asphyxiation, and the tumors were excised and weighed.

When used alone or in combination, TA-1 did not cause any loss of body weight throughout the course of the study, indicating that TA-1 was not toxic.

On Days 3 and 6 only a few mice had palpable tumors, and there was no statistical difference in tumor volume between vehicle control group and any treatment group. On Day 9, most mice of Group 1 (Vehicle Control), Group 2 (DTIC) and Group 3 (TA-1), and only a few of mice in Group 4 (9H10+DTIC) and Group 5 (9H10+DTIC+TA-1) had palpable tumors; the mean tumor volume of Groups 4 and 5 were statistically significantly smaller than Group 1 (p<0.05). On Day 12 and Day 15, all mice in the Groups 1-5 showed palpable tumors, and the mean tumor volume of each drug treatment group except Group 2 (DTIC) was statistically significantly smaller than the vehicle control group (p<0.05).

Based on the tumor size, the tumor volume (TV) was calculated with the formula: [TV=(Length×Width×Width)/2]. And the percent inhibition (PI) of TV (PI_{TV}) was calculated according to the equation below:

PI_{TV}(%)=(TV vehicle−TVdrug treated)/TV vehicle×100

The anti-tumor effect of the test article was further evaluated with tumor weight (TW) measured on day of necropsy (Day 16). The PI of TW was calculated using the equation below:

PI_{TW} (%)=100×(TW vehicle−TW drug treated)/TW vehicle.

On Day 16, the mean tumor weights of all treatment groups were lower than Group 1. The PI_{tw}, values of Group 2, Group 3, Group 4 and Group 5 were 28.41%, 43.35%, 51.35% and 62.05%, respectively, indicating effectiveness of all treatment regimens.

There was no significant difference between each of treatment groups and the vehicle control group on Days 0, 3 and 6. On Days 9, 12, and 15, in comparison to the vehicle group, there were no statistically significant differences in body weights for other groups.

On day 16, tumor weights, were reduced by 43.35% in TA-1-only treated animals. DTIC caused only a modest inhibition in tumor growth (e.g., 28.41%, based on tumor weight), while its combination with 9H10 increased tumor inhibition to 51.35%. When 9H10+DTIC was further combined with TA-1, the tumor inhibition rate was further increased to 62.05%.

### Example 2

### TA1 Plus Ipilimumab

TA1 is administered as a combination therapy to Melanoma patients in treatment regimens at a dosage within a range of 0.5-10 mg/day.

In addition to treatment with TA1, Melanoma patients are treated with ipilimumab in a first protocol and receive ipilimumab at a dose level of 3 mg/kg for all doses in a cohort or an initial loading dose of ipilimumab at 3 mg/kg, which is followed by a subsequent dose at 1 mg/kg in another cohort.

Melanoma patients may be treated with intrapatient ipilimumab dose escalation until objective clinical response, ≧grade 3 autoimmunity or other dose limiting toxicity is reached. Doses may start at 3 mg/kg in cohort 1 or 5 mg/kg in cohort II. Doses may be administered every 3 weeks, escalated to 5 mg/kg or 9 mg/kg every other dose and continued until response, adverse event, or disease progression on 9 mg/kg of antibody is seen.

### Example 3

### TA1 Plus Ticilimumab

TA1 is administered as a combination therapy to Melanoma patients in treatment regimens at a dosage within a range of 0.5-10 mg/day.

In addition to treatment with TA1, Ticilimumab is administered at single dose levels ranging from 0.01 to 15 mg/kg and/or at multiple dose levels ranging from 3 to 15 mg/kg. The dosing regimens may include a single dose or multiple doses given, e.g., q1 month, q3 months, or the like.

1. A method of treating melanoma or a metastasis thereof in a human patient in a combination therapy which comprises administering a melanoma-treating effective combination to a human melanoma patient during a treatment regimen, the combination comprising an alpha thymosin peptide and antibodies against cytotoxic T lymphocyte-associated antigen 4 (CTLA4).

2. The method of claim 1 wherein the CTLA4 antibodies comprise 9H10, MDX010, 1F4, BNI3, Q01, A01, M08, 1B8, WKH203, ab9984, ab13486, ipilimumab, ticilimumab or a combination thereof.

3. The method of claim 1 wherein the CTLA4 antibodies comprise ipilimumab.

4. The method of claim 1 wherein the CTLA4 antibodies comprise ticilimumab.

5. The method of claim 1 wherein the CTLA4 antibodies comprise 9H10.

6. The method of claim 1 wherein said treatment regimen comprises a plurality of days, said alpha thymosin peptide comprises thymosin alpha 1 (TA1), and said TA1 is administered to said patient during at least a portion of said treatment regimen at a dosage within a range of about 0.5-10 mg/day.

7. The method of claim 6 wherein said dosage is within a range of about 1.5-7 mg/day.

8. The method of claim 6 wherein said dosage is within a range of about 3-7 mg/day.

9. The method of claim 6 wherein said dosage is about 3.2 mg/day.

10. The method of claim 6 wherein said dosage is about 6.4 mg/day.

11. The method of claim 1 wherein said alpha thymosin peptide is TA1 and said treatment regimen comprises administration of TA1 daily for a period of about 1-10 days, followed by about 1-5 days of non-administration of said TA1.

12. The method of claim 11 wherein said TA1 is administered daily for about 3-5 days, followed by about 2-4 days of non-administration of said TA1.

13. The method of claim 11 wherein said TA1 is administered daily for about 4 days, followed by about 3 days non-administration of said TA1.

14. The method of claim 1 wherein said antibodies against cytotoxic T lymphocyte-associated antigen 4 (CTLA4) is administered to said patient at a dosage within a range of about 0.001-50 mg/kg patient body weight/day of administration.

15. The method of claim 1 wherein said antibodies against cytotoxic T lymphocyte-associated antigen 4 (CTLA4) is administered to said patient at a dosage of about 0.01-20 mg/kg.

16. The method of claim 1, wherein said combination further includes administration of an alkylating antineoplastic agent (AIkAA).

17. The method of claim 16 wherein the alkylating antineoplastic agent (AIkAA) comprises dacarbazine (DTIC).

18. The method of claim 16 wherein the alkylating antineoplastic agent (AIkAA) is administered to said patient at a dosage within a range of about 700-1300 mg/m²/day.

19. The method of claim 16 wherein the alkylating antineoplastic agent (AIkAA) is administered to said patient at a dosage within a range of about 800-1200 mg/m²/day.
